SEQUENCE LISTING (11 sequences)

SEQ ID NO: 1 - protein, 14 aa, Homo sapiens
RQRREVHEDAHKQV

SEQ ID NO: 2 - protein, 53 aa, Homo sapiens
TESRMHWPGREVHEMSKKGRPQRQRREVHEDAHKQVSPILRRSPDITKSPLTK

SEQ ID NO: 3 - protein, 205 aa, Homo sapiens
SEQLLRIDDHDFSMRPGFGGPAIPVGVDVQVESLDSISEVDMDFTMTLYLRHYWKDERLSFPSTNNLSMTFDGRLVKKIWVPDMFFVHSKRSFIHDTTTDNVMLRVQPDGKVLYSLRVTVTAMCNMDFSRFPLDTQTCSLEIESYAYTEDDLMLYWKKGNDSLKTDERISLSQFLIQEFHTTTKLAFYSSTGWYNRLYINFTLRR

SEQ ID NO: 4 - protein, 13 aa, Homo sapiens
ASMPRVSYIKAVD

SEQ ID NO: 5 - protein, 89 aa, Homo sapiens
TTVQERKEQKLREKLPCTSGLPPPRTAMLDGNYSDGEVNDLDNYMPENGEKPDRMMVQLTLASERSSPQRKSQRSSYVSMRIDTHAIDK

SEQ ID NO: 6 - protein, 473 aa, Homo sapiens
MRFGIFLLWWGWVLATESRMHWPGREVHEMSKKGRPQRQRREVHEDAHKQVSPILRRSPDITKSPLTKSEQLLRIDDHDFSMRPGFGGPAIPVGVDVQVESLDSISEVDMDFTMTLYLRHYWKDERLSFPSTNNLSMTFDGRLVKKIWVPDMFFVHSKRSFIHDTTTDNVMLRVQPDGKVLYSLRVTVTAMCNMDFSRFPLDTQTCSLEIESYAYTEDDLMLYWKKGNDSLKTDERISLSQFLIQEFHTTTKLAFYSSTGWYNRLYINFTLRRHIFFFLLQTYFPATLMVMLSWVSFWIDRRAVPARVPLGITTVLTMSTIITGVNASMPRVSYIKAVDIYLWVSFVFVFLSVLEYAAVNYLTTVQERKEQKLREKLPCTSGLPPPRTAMLDGNYSDGEVNDLDNYMPENGEKPDRMMVQLTLASERSSPQRKSQRSSYVSMRIDTHAIDKYSRIIFPAAYILFNLIYWSIFS

SEQ ID NO: 7 - DNA, 1989 bp, Homo sapiens
cgagaaggatgtttgaatttggaaacccatgttggctgtcccaaatatgagatttggcatctttcttttgtggtggggatgggttttggccactgaaagcagaatgcactggcccggaagagaagtccacgagatgtctaagaaaggcaggccccaaagacaaagacgagaagtacatgaagatgcccacaagcaagtcagcccaattctgagacgaagtcctgacatcaccaaatcgcctctgacaaagtcagaacagcttctgaggatagatgaccatgatttcagcatgaggcctggctttggaggccctgccattcctgttggtgtggatgtgcaggtggagagtttggatagcatctcagaggttgacatggactttacgatgaccctctacctgaggcactactggaaggacgagaggctgtcttttccaagcaccaacaacctcagcatgacgtttgatggccggctggtcaagaagatctgggtccctgacatgtttttcgtgcactccaaacgctccttcatccacgacaccaccacagacaacgtcatgttgcgggtccagcctgatgggaaagtgctctatagtctcagggttacagtaactgcaatgtgcaacatggacttcagccgatttcccttggacacacaaacgtgctctcttgaaattgaaagctatgcctatacagaagatgacctcatgctgtactggaaaaagggcaatgactccttaaagacagatgaacggatctcactctcccagttcctcattcaggaattccacaccaccaccaaactggctttctacagcagcacaggctggtacaaccgtctctacattaatttcacgttgcgtcgccacatcttcttcttcttgctccaaacttatttccccgctaccctgatggtcatgctgtcctgggtgtccttctggatcgaccgcagagccgtgcctgccagagtccccttaggtatcacaacggtgctgaccatgtccaccatcatcacgggcgtgaatgcctccatgccgcgcgtctcctacatcaaggccgtggacatctacctctgggtcagctttgtgttcgtgttcctctcggtgctggagtatgcggccgtcaactacctgaccactgtgcaggagaggaaggaacagaagctgcgggagaagcttccctgcaccagcggattacctccgccccgcactgcaatgctggacggcaactacagtgatggggaggtgaatgacctggacaactacatgccagagaatggagagaagcccgacaggatgatggtgcagctgaccctggcctcagagaggagctccccacagaggaaaagtcagagaagcagctatgtgagcatgagaatcgacacccacgccattgataaatactccaggatcatctttccagcagcatacattttattcaatttaatatactggtctattttctcctagatgcttgtaattctacaaatttcacatttccatggcatgcactacagaaataactgtataatgaaaaagtatttaaggatatggttaaaaaaaaatcccaggacccacccatgttttcactatcccttctgcagctttccaaagctacattgacgagacacttactggtttaatttgcacttattaaccgtctgttgaatacacagcattatattaggtgctgcagaaatacgacactgtagcgactgatgttagttgttacccagataaaatggaaaagcacactaccagtgttgtgggcacatttagytccacccgattagacccttgatgctattcacatgaataatttatttttccctaaaagtgtcattacattgttcaggctacgtgaacttggaagcaccatcaggccatttgcatgaaattcacatgcacctaaatcctcactttgacagaaactcatgcttcagttataacctattacctattttgtatgcgactccacctccgcatgttcg

SEQ ID NO: 8 - protein, 24 aa, Homo sapiens
HIFFFLLQTYFPATLMVMLSWVSF

SEQ ID NO: 9 - protein, 24 aa, Homo sapiens
AVPARVPLGITTVLTMSTIITGVN

SEQ ID NO: 10 - protein, 23 aa, Homo sapiens
IYLWVSFVFVFLSVLEYAAVNYL

SEQ ID NO: 11 - protein, 20 aa, Homo sapiens
YSRIIFPAAYILFNLIYWSI

(19) United States
(12) Patent Application Publication
(10) Pub. No.: US 2011/0229909 A1
(43) Pub. Date: Sep. 22, 2011

(54) RHO1-Gamma Amino Butyric Acid C Receptor-Specific Antibodies

(75) Inventors: David R. Pepperberg, Chicago, IL (US); Helene A. Gussin, Northbrook, IL (US); Fadi T. Khasawneh, Upland, CA (US); An Xie, Hinsdale, IL (US); Haohua Qian, Lisle, IL (US); Guy C. Le Breton, Oak Park, IL (US)

(73) Assignee: The Board of Trustees of the University of Illinois, Urbana, IL (US)

(21) Appl. No.: 13/151,578

(22) Filed: Jun. 2, 2011

Related U.S. Application Data
(63) Continuation of application No. 12/430,716, filed on Apr. 27, 2009, now abandoned.
(60) Provisional application No. 61/047,946, filed on Apr. 25, 2008.
(60) Provisional application No. 61/125,570, filed on Apr. 25, 2008.

(51) Int. Cl. G01N 33/53; C07K 16/28

(52) U.S. Cl. 435/7.1; 530/389.1; 530/388.22; 436/501

(57) ABSTRACT

This invention provides antibodies immunologically specific for ρ1-GABA_{C} receptor protein. The invention also provides methods of making and methods of using said antibodies and kits containing the antibodies.

This application claims the benefit of priority to U.S. provisional application Ser. Nos. 61/047,946 and 61/125,570, both of which were filed on Apr. 25, 2008. The disclosures of both provisional applications are incorporated herein by reference in their entireties.

This invention was supported in part by grants (Nos. EY016094, EY001792, and HL024530) from the National Institutes of Health, National Eye Institute. The government has certain rights in this invention.

### BACKGROUND OF THE INVENTION

1. Field of the Invention

This invention relates to antibodies immunologically specific for Rho1-gamma amino butyric acid C (ρ1-GABA_{C}) receptor protein. The invention particularly relates to polyclonal antisera, monoclonal antibodies and fragments and derivatives thereof that are immunologically specific for ρ1-GABA_{C} receptor. Methods for making and using said antibodies are also provided.

2. Summary of the Related Art

Gamma-aminobutyric acid (GABA) is the main inhibitory neurotransmitter found in the central nervous system and retina. The GABA_{C} receptor, a ligand-gated ion chloride channel, is expressed in many areas of the brain, with especially high expression levels in the retina (Qian et al. 1994 J. Neurosci. 14:4299-4307; Enz et al., 1996 J. Neurosci. 16:4479-90; Euler et al., 1998 J. Neurophysiol. 79:1384-95; Lukasiewicz et al., 1998 J. Neurophysiol. 79:3157-67; reviewed by Lukasiewicz, 2005 Prog. Brain Res. 147:205-18). The functional GABA_{C} receptor is formed by oligomerization of five subunits, with ligand binding sites located at the junction between subunits on the long N-terminal extracellular domain, and a central channel (Amin et al., 1996 Proc. R. Soc. 263, 273-282). Native GABA_{C} receptors consist of different subunits, e.g. for human ρ1 and ρ2, and for rat ρ1, ρ2, ρ3, etc. One of the most studied GABA_{C} receptors, that of rodent retinal bipolar cells, consists mostly of heteromers of at least ρ1 and ρ2 subunits (Zhang et al., 1995 Proc. Natl. Acad. Sci. USA 92: 11756-11760). However, the ρ1 subunit can assemble to form functional homopentameric receptors (Qian et al., 1998 J. Neurobiol. 37:305-320).

The inhibitory action mediated by the gated chloride channel of GABA_{C}-R can control glutamate neurotransmitter release from retinal bipolar cells, and lessen the activity of inner retinal neurons. Reducing the level of neuronal excitability by activating GABA_{C}-R in the retina can be beneficial for preserving visual function under certain pathological conditions. For example, glaucoma, whose clinical hallmark is the loss of retinal ganglion cells, is thought to be caused in large part by glutamate-induced excitotoxicity (Qian, et al., 2008, Exp. Eye Res. doi:10.1016/j.exer.2008.10.005). On the other hand, GABA_{C}-R antagonists have been implicated in the prevention of form-deprivation-induced myopia. Thus, GABA_{C}-R is a potential target for various ocular disorders. The availability of an antibody directed against the ρ1 GABA_{C} receptor, that exhibits specificity and high affinity, would be an asset for further study of the GABA_{C} receptor, and for diagnostic or therapeutic uses relating to diseases and disorders involving the receptor or ligands thereof.

### SUMMARY OF THE INVENTION

In one aspect, this invention provides antibodies that specifically bind to ρ1-GABA_{C} receptor. In certain embodiments, the antibodies comprise a polyclonal antisera. In alternative embodiments, the antibody is a monoclonal antibody. In certain preferred embodiments, the antibodies of the invention specifically bind to an epitope defined by an amino acid sequence identified by SEQ ID NO: 1. Antibodies of the invention are advantageously produced by immunizing an animal with a peptide having the amino acid sequence as identified by SEQ ID NO: 1. In other aspects, the invention provides methods for detecting ρ1-GABA_{C} receptor comprising the steps of contacting a sample comprising ρ1-GABA_{C} receptor with an antibody of the invention and detecting binding of the antibody with the protein. In particular, ρ1-GABA_{C} can be detected in retinal cells and tissues and in certain brain tissues.

In certain aspects, the invention also provides methods for detecting ρ1-GABA_{C} expression, particularly in retinal cells and tissues and in certain brain tissues using the antibodies of the invention. ρ1-GABA_{C} receptor can be detected using methods including without limitation in situ immunohistochemistry and Western blot analysis.

The invention also provides a kit for practicing the methods of the invention, comprising a preparation of the antibodies of the invention and instructions for use. In certain embodiments, the kits also contain reagents, such as reagents for in situ hybridization, useful in the practice of the methods of the invention. In certain other embodiments, the kit further comprises a control sample or a standard.

Specific preferred embodiments of the present invention will become evident from the following more detailed description of certain preferred embodiments and the claims.

### BRIEF DESCRIPTION OF THE DRAWINGS

FIG. 1 shows the amino acid sequence of the human ρ1 GABA_{C} receptor (SEQ ID NO:6). The signal peptide consists of residues 1-15, the “unstructured” amino-terminal sequence of the mature protein consists of residues 16-68 (SEQ ID NO:2), wherein the sequence of the target peptide (SEQ ID NO: 1) is in bold and underlined text; the core domain consists of residues 69-273 (SEQ ID NO: 3) and the transmembrane domain is represented by residues 274-297 (SEQ ID NO:8), 303-326 (SEQ ID NO:9), 340-362 (SEQ ID NO:10), and 452-471 (SEQ ID NO:11).

FIGS. 2A through 2D show the results of immunoblotting experiments.

FIG. 2A shows the results of spot-blot assays, using 1 ng (top) and 0.1 ng (bottom) of N-14 peptide (SEQ ID NO:1) spotted on the membrane. Lane 1: peptide probed with affinity-purified GABA_{C} Ab N-14. Lane 2: peptide probed only with the secondary antibody (i.e., affinity-purified GABA_{C} Ab N-14 omitted).

FIG. 2B shows the results of Western-blot assay performed using whole-cell lysates of GABA_{C}-expressing and non-GABA_{C} expressing neuroblastoma cells. Lane 1: Test of SHp5-ρ1 neuroblastoma cells, which were genetically engineered to express human GABA_{C}. Cells were probed with affinity-purified GABA_{C} Ab N-14 (as described herein), followed by the secondary antibody. Lane 2: Test of SHSY5Y control neuroblastoma cells (which do not express GABA_{C}), probed with the affinity-purified GABA_{C} Ab N-14, followed by the secondary antibody. Lane 3: GABA_{C}-expressing SHp5-ρ1 probed only with the secondary antibody (affinity-purified GABA_{C} Ab N-14 omitted). Lane 4: Pre-absorption control. GABA_{C}-expressing SHp5-ρ1 cells probed with affinity-purified GABA_{C} Ab N-14 that had been pre-absorbed with the N-14 cognate peptide (3 μg/ml, 30 min, RT), followed by the secondary antibody.

FIG. 2C shows the results of Western blot assays performed using Xenopus laevis oocytes generically engineered to express GABA_{C}. Lanes 1, 3 and 4: Membrane preparations obtained from GABA_{C}-expressing oocyte. Lane 2: Non-expressing control oocyte. Experimental conditions used for lanes 1-4 are otherwise identical to those of panel B.

FIG. 2D shows the results of Western blot assay performed on whole cell lysates of rat brain and rat retina, probed with (1) the affinity-purified GABA_{C} Ab N-14 followed by the secondary antibody; (2) with the secondary antibody only (i.e., affinity-purified GABA_{C} Ab N-14 omitted); and (3) the affinity-purified GABA_{C} Ab N-14 pre-absorbed with the N-14 cognate peptide (3 μg/ml, 30 min, RT), followed by the secondary antibody.

FIG. 3 shows the results of Western-blot assay performed using oocytes. Lane 1: Membrane preparations obtained from GABA_{A}-expressing oocytes. Lane 2: Membrane preparations obtained from non-expressing oocytes. Lane 3: Membrane preparations obtained from GABA_{C}-expressing oocytes. The preparations were probed with affinity-purified GABA_{C} Ab N-14, followed by the secondary antibody.

FIGS. 4A and 4B show the results of flow cytometry analysis of GABA_{C}-expressing neuroblastoma cells (SHp5-ρ1) and non-expressing controls (SHSY5Y), using affinity-purified GABA_{C} Ab N-14. M1 region: FIG. 4A is a flow cytometric profile of SHp5-ρ1 cells, probed with non-immune guinea pig IgG as a primary antibody. FIG. 4B shows flow cytometry of non-expressing cells probed with GABA_{C} Ab N-14 as a primary antibody. M2 region (FIGS. 4A and 4B): Profile of SHp5-ρ1 cells probed with the affinity-purified GABA_{C} Ab N-14 at dilutions of 1/25; 1/50, and 1/1,000. The 1/25 or 1/50 dilutions resulted in ˜63% positive cells, and the 1/1,000 dilution resulted in ˜47% positive cells.

FIGS. 5A and 5B show the results of immunofluorescence assays of GABA_{C} expressing and non-expressing neuroblastoma cells. In FIG. 5A, Panel 1 shows the results of incubating SHp5-ρ1 cells for 1 hr with affinity-purified GABA_{C} Ab N-14 (1/1000), followed by a 45-min incubation with biotinylated secondary antibody, and 1-hr incubation with streptavidin-conjugated quantum dots. Arrows indicate positive immunofluorescence staining at the cell surface using GABA_{C} Ab N-14. In Panel 2, conditions were as in Panel 1, but with omission of the affinity-purified GABA_{C} Ab N-14. In Panel 3, non-expressing SHSY5Y cells were incubated with affinity-purified GABA_{C} Ab N-14, followed by the biotinylated secondary antibody, and by streptavidin-conjugated quantum dots (dilutions and incubation periods as in Panel 1).

FIG. 5B shows the electrophysiological response of SHp5-ρ1 cells induced by 10 μM GABA measured in picoamperes (left: current traces; right: peak current amplitudes). The numbers within the bars of the bar graph (from left to right: “9”, “8”, and “10”) indicate the number of experiments for each subgroup. Control cells were not treated with antibody. “1st Ab” represents SHp5-ρ1 cells that were incubated for 1 hr with affinity-purified GABA_{C} Ab N-14 (1/1,000) alone. “1st Ab+Biotin+QD” represents SHp5-ρ1 cells that were incubated for 1 hr with affinity-purified GABA_{C} Ab N-14 (1/1,000) followed by a 45-min incubation with biotinylated secondary antibody, and a 1-hour incubation with streptavidin-conjugated quantum dots. For all measurements, the holding potential was −60 mV.

### DETAILED DESCRIPTION OF THE PREFERRED EMBODIMENTS

In one aspect, the invention provides antibodies, including polyclonal antisera, monoclonal antibodies and antigen-binding fragments and derivatives thereof, that are immunologically specific for ρ1-GABA_{C}. These antibodies are prepared to be immunologically specific for a peptide antigen comprising a portion of the amino acid sequence of human ρ1-GABA_{C}. This peptide antigen is identified by the sequence:
RQRREVHEDAHKQV (SEQ ID NO: 1).

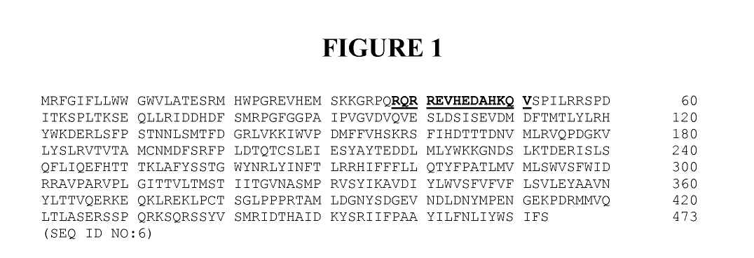

The amino acid sequence of human ρ1-GABA_{C} is shown in FIG. 1. The 14-mer peptide (N-14) identified by SEQ ID NO:1 is located within the N-terminal region of the human ρ1 subunit. There are specific features of this sequence that comprised at least a portion of the selection criteria for choosing this peptide fragment for antibody production. These include that it is not part of the “core peptide”, i.e., not part of the more conserved region believed to be involved in inter-subunit interaction, ligand binding, and channel formation (when the sequence of the core peptide was analyzed using the NCBI BLAST/Blastp server, a neurotransmitter gated ion-channel ligand binding domain was detected). Also, the sequence is located in the “unstructured tail” of the N-terminal region, which is less conserved among species (when the sequence of the “unstructured tail” region was analyzed using the BLAST/Blastp server, no putative conserved domains were detected). In addition, a computer search for the selected N-14 sequence using the ExPaSy and NCBI websites (computation performed at the SIB using the BLAST network service), yielded the following matches: human ρ1 GABA_{C} (14/14), rat ρ1 GABA_{C} (11/14), mouse ρ1 GABA_{C} (10/14), and Burkholderia phymatum (a proteobacteria) hydrolase (9/14), consistent with the antigen defined by N-14 being specific for human ρ1 GABA_{C}.

Either the full-length ρ1-GABA_{C} protein or peptide fragments thereof can be used as antigens for generating ρ1-GABA_{C}-specific antibodies. In certain embodiments, the peptides used as antigen is 10-300, 100-200, 100-150, 10-15, 10-50, 20-30 or 50-150 amino acid residues in length. In one preferred embodiment, antibodies are generated using the peptide of amino acid residues 38-51 of full-length ρ1-GABA_{C} (i.e., SEQ ID NO:1) as an antigen. The skilled worker will understand that antibodies generated using a peptide fragment of the full length ρ1-GABA_{C} as antigen can recognize and specifically bind to the full-length ρ1-GABA_{C}.

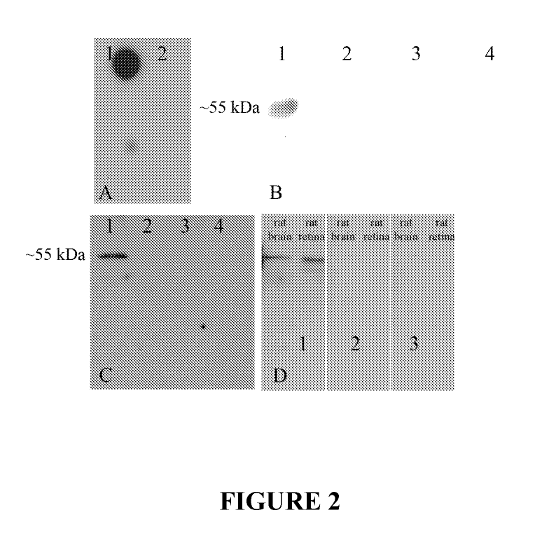

It will be understood in the art that antigenic peptides provided herein each form an epitope that is recognized by said immunologically-specific antibodies of the invention, wherein the peptide epitope is in a configuration that is sufficiently structurally equivalent to the configuration of this amino acid sequence in the native ρ1-GABA_{C} protein. The immunological specificity of antibodies of this invention is shown herein in FIGS. 2 through 5 as described in more detail below. As used herein, the term “immunologically specific” is intended to mean that the antibodies of this invention specifically bind to the ρ1-GABA_{C} type of protein without significantly detectable cross-reactivity to any other GABA receptor types.

Antibodies of the invention can be produced by any method known in the art for the synthesis of antibodies, including chemical synthesis or recombinant expression techniques, or preferably using conventional immunological methods. As used herein, the term “antibody” includes, but is not limited to, both naturally occurring and non-naturally occurring antibodies. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. More specifically, the term “antibody” includes polyclonal antisera and monoclonal antibodies, and antigen-binding fragments thereof such as Fab, Fab′, and F(ab′)₂ fragments. Furthermore, the term “antibody” includes chimeric antibodies and wholly synthetic antibodies, including genetically engineered antibodies, and fragments thereof. The polyclonal and monoclonal antibodies may be “purified” which means the polyclonal and monoclonal antibodies are free of any other antibodies.

The N-14 epitope peptide (SEQ ID NO: 1) disclosed herein is advantageously used to prepare antibodies that specifically bind to ρ1-GABA_{C}. The affinity of a monoclonal antibody can be readily determined by one of ordinary skill in the art (see, for example, ANTIBODIES: A LABORATORY MANUAL, Harlow and Lane (eds.), Cold Spring Harbor Laboratory Press, 1988).

Methods generally used for recombinant DNA technologies and methods for preparing polyclonal and monoclonal antibodies are well known in the art (see for example, Sambrook et al., 1989, MOLECULAR CLONING: A LABORATORY MANUAL, Second Edition, Cold Spring Harbor, N.Y.; and Hurrell (Ed.), MONOCLONAL HYBRIDOMA ANTIBODIES: TECHNIQUES AND APPLICATIONS, CRC Press, Inc., Boca Raton, Fla., 1982, which are incorporated herein by reference). As would be evident to one of ordinary skill in the art, polyclonal antibodies can be generated from a variety of warm-blooded animals such as horses, cows, goats, sheep, dogs, chickens, rabbits, mice, and rats, and in certain embodiments as disclosed herein, guinea pigs. The immunogenicity of the ρ1-GABA_{C} N-14 epitope peptide (SEQ ID NO: 1) as disclosed herein can be increased through the use of an adjuvant such as Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. Information concerning adjuvants and various aspects of immunoassays are disclosed, for example, in Tijssen (1987, PRACTICE AND THEORY OF ENZYME IMMUNOASSAYS, 3rd Ed., Elsevier: New York). Other useful references covering methods for preparing polyclonal antisera include MICROBIOLOGY (1969, Hoeber Medical Division, Harper and Row); Landsteiner (1962, SPECIFICITY OF SEROLOGICAL REACTIONS, Dover Publications: New York), and Williams et al. (1967, METHODS IN IMMUNOLOGY AND IMMUNOCHEMISTRY, Vol. 1, Academic Press: New York).

As is well known in the art, a given composition may vary in its immunogenicity. Peptide antigen fragments may be joined to other materials, particularly polypeptides, as fused or covalently joined polypeptides to be used as immunogens. An antigen and its fragments may be fused or covalently linked to a variety of immunogens, such as keyhole limpet hemocyanin (KLH), bovine serum albumin (BSA) and other albumins such as ovalbumin, mouse serum albumin or rabbit serum albumin, tetanus toxoid, etc. Means for conjugating a polypeptide to a carrier protein are well known in the art and include glutaraldehyde, m-maleimidobenzoyl-N-hydroxysuccinimide ester, carbodiimide and bis-biazotized benzidine. See Microbiology, Hoeber Medical Division, Harper and Row, 1969; Landsteiner, 1962, Specificity of Serological Reactions, Dover Publications, New York; Williams et al., 1967, Methods in Immunology and Immunochemistry, vol. 1, Academic Press, New York; and Harlow and Lane, 1988, Id., for descriptions of methods of preparing polyclonal antisera.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored.

Serum produced from animals immunized using standard methods can be used directly, or the IgG fraction can be separated from the serum using standard methods such as plasmaphoresis or adsorption chromatography with IgG-specific adsorbents such as immobilized Protein A.

Antibody fragments, such Fab, Fab′, and F(ab′)₂ fragments, can be produced from the corresponding antibodies by cleavage of and collection of the desired fragments in accordance with known methods (see, for example, Andrew et al., 1992, “Fragmentation of Immunoglobulins” in CURRENT PROTOCOLS IN IMMUNOLOGY, Unit 2.8, Greene Publishing Assoc. and John Wiley & Sons).

A variety of assays known to those skilled in the art can be utilized to detect antibodies which specifically bind to full-length ρ1-GABA_{C} protein or a ρ1-GABA_{C} epitope peptide of this invention, in particular the N-14 peptide identified by SEQ ID NO:1. Exemplary assays are described in detail in Harlow & Lane. (1988, Id.). Representative examples of such assays include: concurrent immunoelectrophoresis, radio-immunoassays, radio-immunoprecipitations, enzyme-linked immunosorbent assays (ELISA), dot blot assays, Western blot assays, inhibition or competition assays, and sandwich assays.

Alternatively, monoclonal antibodies against the antigenic peptides of the invention can be prepared according to well-known techniques, such as those exemplified in U.S. Pat. No. 4,196,265, incorporated herein by reference in its entirety. Hybridomas producing monoclonal antibodies against the antigenic peptides of the invention are produced by well-known techniques. Usually, the process involves the fusion of an immortalizing cell line with a B-lymphocyte that produces the desired antibody. Immortalizing cell lines are usually transformed mammalian cells, particularly myeloma cells of rodent, bovine, and human origin. Rodents such as mice and rats are preferred animals, however, the use of rabbit or sheep cells is also possible. Mice are preferred, with the BALB/c mouse being most preferred as this is most routinely used and generally gives a higher percentage of stable fusions.

Techniques for obtaining antibody-producing lymphocytes from mammals injected with antigens are well known. Generally, peripheral blood lymphocytes (PBLs) are used if cells of human origin are employed, or spleen or lymph node cells are used from non-human mammalian sources. A host animal is injected with repeated dosages of the purified antigen, and the animal is permitted to generate the desired antibody-producing cells before they are harvested for fusion with the immortalizing cell line. Most frequently, immortalized cell lines are rat or mouse myeloma cell lines that are employed as a matter of convenience and availability. Techniques for fusion are also well known in the art, and in general involve mixing the cells with a fusing agent, such as polyethylene glycol.

Generally, following immunization somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately fifty million to two hundred million lymphocytes.

Myeloma cell lines are suited for use in hybridoma-producing fusion procedures and preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art. Available murine myeloma lines, such as those from the American Type Culture Collection (ATCC), 10801 University Boulevard, Manassas, Va. 20110-2209, USA, may be used in the hybridization. For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions. One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler et al., 1975, Nature 256:495; Kohler et al., 1976, Eur. J. Immunol. 6:511; Kohler et al., 1976, Eur. J. Immunol. 6:292), and those using polyethylene glycol (PEG), such as 37% (v/v) PEG (Gefter et al., 1977, Somatic Cell Genet. 3:231-236). The use of electrically induced fusion methods is also appropriate (Goding, 1986, Monoclonal antibodies: Principles and Practice, pp. 60-74, 2nd Edition, Academic Press, Orlando, Fla.).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. The preferred selection medium is HAT. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

Culturing the fusion products under these conditions provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. Hybridomas secreting the desired antibody are selected using standard immunoassays, such as Western blotting, ELISA (enzyme-linked immunosorbent assay), RIA (radioimmunoassay), or the like. Antibodies are recovered from the medium using standard protein purification techniques (such as Tijssen, Id.). The assay should be sensitive, simple and rapid, such as radioimmunoassay, enzyme immunoassays, cytotoxicity assays, plaque assays, dot immunobinding assays, and the like.

The selected hybridomas are then serially diluted and cloned into individual antibody-producing cell lines, which clones can then be propagated indefinitely to provide mAbs. The cell lines may be exploited for mAb production in at least two ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

Many references are available to provide guidance in applying the above techniques, including Kohler et al. (1980, HYBRIDOMA TECHNIQUES, Cold Spring Harbor Laboratory, New York); Tijssen (Id.); Campbell (1984, MONOCLONAL ANTIBODY TECHNOLOGY, Elsevier: Amsterdam); Hurrell (1982, Id.). Monoclonal antibodies can also be produced using well known phage library systems. See, for example, Huse et al. 1989, Science 246:1275; Ward et al. 1989, Nature 341:544.

Antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fᵥ or disulfide stabilized Fᵥ antibody domains recombinantly fused to either the phage gene III or gene VIII protein.

Examples of phage display methods that can be used to make the antibodies of the present invention include those disclosed in Brinkman et al. (1995, J. Immunol. Methods 182:41-50); Ames et al. (1995, J. Immunol. Meth. 184:177-186); Kettleborough et al. (1994, Eur. J. Immunol. 24:952-958); Persic et al. (1997, Gene 187:9-18); Burton et al. (1994, Adv. Immunol. 57:191-280); PCT publication No. WO1992/001047; PCT publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can also be employed using methods known in the art such as those disclosed in PCT publication WO 92/22324; Mullinax et al. (1992, BioTechniques 12:864-869); Sawai et al. (1995, AJRI 34:26-34); and Better et al. (1988, Science 240:1041-1043), said references being incorporated by reference in their entireties.

Examples of techniques which can be used to produce single-chain Fᵥs and antibodies include those described in U.S. Pat. Nos. 4,946,778 and 5,258,498; Huston et al. (1991, Methods in Enzymology 203:46-88); Shu et al. (1993, Proc. Natl. Acad. Sci. USA 90:7995-7999); and Skerra et al. (1998, Science 240:1038-1040).

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison (1985, Science 229:1202); Oi et al. (1986, BioTechniques 4:214); Gillies et al. (1989, J. Immunol. Methods 125:199-202); U.S. Pat. Nos. 5,807,715; 4,816,567; and 4,816,397, which are incorporated herein by reference in their entireties.

Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, for example, U.S. Pat. No. 5,585,089, and Riechmann et al., 1988, Nature 332:323, which are incorporated herein by reference in their entireties.) Antibodies can be humanized using a variety of techniques known in the art including, for example, CR-grafting (European Patent Application, Publication No. EP239400; PCT publication No. WO 91/09967; U.S. Pat. Nos. 5,225,539; 5,530,101; and 5,585,089), veneering or resurfacing (European Patent Applications, Publication Nos. EP592106; EP519596; Padlan, 1991, Molecular Immunology 28:489 498; Studnicka et al., 1994, Protein Engineering 7: 805 814; Roguska et al., 1994, Proc. Natl. Acad. Sci. USA 91:969-973), and chain shuffling (U.S. Pat. No. 5,565,332). Completely human antibodies are particularly desirable for therapeutic treatment of human patients. Human antibodies can be made by a variety of methods known in the art including phage display methods using antibody libraries derived from human immunoglobulin sequences. See also, U.S. Pat. Nos. 4,444,887 and 4,716,111; and PCT publications Nos. WO 98/46645, WO 98/50433, WO 98/24893, WO 98/16654, WO 96/34096, WO 96/33735, and WO 91/10741; each of which is incorporated herein by reference in its entirety. Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., 1988, Biotechnology 12:899-903).

Examples of techniques which can be used to produce single-chain Fᵥs and antibodies include those described in U.S. Pat. Nos. 4,946,778 and 5,258,498; Huston et al. (1991, Methods in Enzymology 203:46-88); Shu et al. (1993, Proc. Natl. Acad. Sci. USA 90:7995-7999); and Skerra et al. (1998, Science 240:1038-1040).

In addition, techniques developed for the production of “chimeric antibodies” (Morrison et al., 1984, Proc. Natl. Acad. Sci. USA 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, 1988, Science 242:423-42; Huston et al., 1988, Proc. Natl. Acad. Sci. USA 85:5879 5883; and Ward et al., 1989, Nature 334:544-54) can be adapted to produce single chain antibodies specific for ρ1-GABA_{C}. Single chain antibodies are formed by linking the heavy and light chain fragments of the Fᵥ region via an amino acid bridge, resulting in a single chain polypeptide. Techniques for the assembly of functional Fᵥ fragments in E. coli may also be used (Skerra et al., 1988, Science 242:1038 1041).

Recombinant expression of an antibody of the invention, or fragment, derivative or analog thereof (e.g., a heavy or light chain of an antibody of the invention or a single chain antibody of the invention) requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein.

Methods well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, for example, PCT Publication Nos. WO86/05807, WO 89/01036; and U.S. Pat. No. 5,122,464) and the variable domain of the antibody may be cloned into such a vector for expression of the entire heavy or light chain.

Expression vectors as disclosed herein are transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed herein.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

Preferably, bacterial cells such as E. coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; Cockett et al., 1990, Bio/Technology 8:2).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye et al., 1985, Nucleic Acids Res. 13:3101-3109; Van Heeke et al., 1989 J. Biol. Chem. 264:5503-5509); and the like. pGEX vectors (Stratagene, LaJolla, Calif.) may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems maybe utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (See, for example, Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, and other elements (see Bittner et al., 1987, Methods in Enzymol. 153:515-44).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter and enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., 1977, Cell 11:223), hypoxanthine-guanine phosphoribosyltransferase (Szybalska & Szybalski, 1992, Proc. Natl. Acad. Sci. USA 48:202), and adenine phosphoribosyltransferase (Lowy et al., 1980, Cell 22:817) genes can be employed in TK-, HGPRT- or APRT-cells, respectively. Also, antimetabolite resistance can be used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Mulligan, 1993, Science 260:926-932); and hyg, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Id.; Kriegler, 1990, GENE TRANSFER AND EXPRESSION, A LABORATORY MANUAL, Stockton Press, NY; and Colberre-Garapin et al., 1981, J. Mol. Biol. 150:1, which are incorporated by reference herein in their entireties.

The expression levels of an antibody molecule can be increased by vector amplification. When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197; and U.S. Pat. Nos. 4,816,567, 6,331,415, all references being incorporated by references in their entireties). The coding sequences for the heavy and light chains may comprise cDNA or genomic DNA.

Once an antibody molecule of the invention has been produced by any method disclosed herein or known in the art, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

Antibodies thus produced, whether polyclonal or monoclonal, can be used, e.g., in an immobilized form bound to a solid support by well known methods.

Antibodies against the antigenic peptides of the invention can also be used, unlabeled or labeled by standard methods, as the basis for immunoassays and immunospecific binding to ρ1-GABA_{C}. The immunoassays which can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., Eds, 1994, Id.). In particular, the antibodies of the present invention may also be used in conjunction with both fresh-frozen and/or formalin-fixed, paraffin-embedded tissue blocks prepared for study by immunohistochemistry (IHC). For example, immunohistochemistry may be utilized to evaluate tumor tissue for expression of ρ1-GABA_{C} species.

Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions that can be conjugated to antibodies for use as diagnostics according to the present invention. The particular label used will depend upon the type of immunoassay. Examples of labels that can be used include but are not limited to radiolabels such as 3H, 14C, ³²P, ¹²⁵I, ¹³¹I, ¹¹¹In or ⁹⁹Tc; fluorescent labels such as fluorescein and its derivatives, rhodamine and its derivatives, dansyl and umbelliferone; chemiluminescers such as luciferase and 2,3-dihydro-phthalazinediones; and enzymes such as horseradish peroxidase, alkaline phosphatase, lysozyme, glucose-6-phosphate dehydrogenase, and acetylcholinesterase. The antibodies can be tagged with such labels by known methods. For example, coupling agents such as aldehydes, carbodiimides, dimaleimide, imidates, succinimides, bisdiazotized benzadine and the like may be used to tag the antibodies with fluorescent, chemiluminescent or enzyme labels. The general methods involved are well known in the art and are described, for example, in IMMUNOASSAY: A PRACTICAL GUIDE (1987, Chan (Ed.), Academic Press, Inc.: Orlando, Fla.). An alternative to labeling an antibody produced according to this invention is to use a labeled, secondary antibody specific for the immunoglobulin species and subtype produced according to the methods of the invention (using, for example, goat anti-guinea pig IgG antibody). Such methods are well known in the art (Id.).

The invention also provides a kit containing an antibody of the invention, preferably conjugated to a detectable substance, and instructions for use.

It is understood that the peptide portion of ρ1-GABA_{C} protein used as an antigen for raising the antibodies of the invention (N-14 peptide, identified as SEQ ID NO:1) comprises an epitope that defines the chemical and three-dimensional structure of these antibodies. This antigenic epitope is understood in the art as comprising a three-dimensional structure that defines the immunological activity of the epitope. Peptides as identified by the invention can be advantageously synthesized by any of the chemical synthesis techniques known in the art, particularly solid-phase synthesis techniques, for example, using commercially-available automated peptide synthesizers (see, for example, Merrifield, 1963, J. Amer. Chem. Soc. 85: 2149-54; Carpino, 1973, Acc. Chem. Res. 6: 191-98; Birr, 1978, ASPECTS OF THE MERRIFIELD PEPTIDE SYNTHESIS, Springer-Verlag: Heidelberg; THE PEPTIDES: ANALYSIS, SYNTHESIS, BIOLOGY, Vols. 1, 2, 3, 5, (Gross & Meinhofer, eds.), Academic Press: New York, 1979; Stewart et al., 1984, SOLID PHASE PEPTIDE SYNTHESIS, 2nd. ed., Pierce Chem. Co.: Rockford, Ill.; Kent, 1988, Ann. Rev. Biochem. 57: 957-89; and Gregg et al., 1990, Int. J. Peptide Protein Res. 55: 161-214, which are incorporated herein by reference in their entirety.) Alternatively, the antigen peptide can be recombinantly produced using methods well known in the art. The recombinantly produced peptides can be affinity-purified by way of an engineered epitope tag, such as a His-tag or a GST-tag. The antigen peptide can be freed from the epitope tag by proteolytic cleavage at a protease cleavage site engineered between the epitope tag and the antigen peptide. The coding sequence of ρ1-GABA_{C} is known in the art and further shown in nucleotides 47 to 1468 of SEQ ID NO:7.

The use of solid phase methodology is preferred. Briefly, an N-protected C-terminal amino acid residue is linked to an insoluble support such as divinylbenzene cross-linked polystyrene, polyacrylamide resin, Kieselguhr/polyamide (pepsyn K), controlled pore glass, cellulose, polypropylene membranes, acrylic acid-coated polyethylene rods or the like. Cycles of deprotection, neutralization and coupling of successive protected amino acid derivatives are used to link the amino acids from the C-terminus according to the amino acid sequence. For some synthetic peptides, an FMOC strategy using an acid-sensitive resin may be used. Preferred solid supports in this regard are divinylbenzene cross-linked polystyrene resins, which are commercially available in a variety of functionalized forms, including chloromethyl resin, hydroxymethyl resin, paraacetamidomethyl resin, benzhydrylamine (BHA) resin, 4-methylbenzhydrylamine (MBHA) resin, oxime resins, 4-alkoxybenzyl alcohol resin (Wang resin), 4-(2′,4′-dimethoxyphenylaminomethyl)-phenoxymethyl resin, 2,4-dimethoxybenzhydryl-amine resin, and 4-(2′,4′-dimethoxyphenyl-FMOC-amino-methyl)-phenoxyacetamidonorleucyl-MBHA resin (Rink amide MBHA resin). In addition, acid-sensitive resins also provide C-terminal acids, if desired. A particularly preferred protecting group for alpha amino acids is base-labile 9-fluorenylmethoxy-carbonyl (FMOC).

Suitable protecting groups for the side chain functionalities of amino acids chemically compatible with BOC (t-butyloxycarbonyl) and FMOC groups are well known in the art. When using FMOC chemistry, the following protected amino acid derivatives are preferred: FMOC-Cys(Trit), FMOC-Ser(But), FMOC-Asn(Trit), FMOC-Leu, FMOC-Thr(Trit), FMOC-Val, FMOC-Gly, FMOC-Lys(Boc), FMOC-Gln(Trit), FMOC-Glu(OBut), FMOC-His(Trit), FMOC-Tyr(But), FMOC-Arg(PMC (2,2,5,7,8-pentamethylchroman-6-sulfonyl)), FMOC-Arg(BOC)₂, FMOC-Pro, and FMOC-Trp(BOC). The amino acid residues can be coupled by using a variety of coupling agents and chemistries known in the art, such as direct coupling with DIC (diisopropyl-carbodiimide), DCC (dicyclohexylcarbodiimide), BOP (benzotriazolyl-N-oxytrisdimethylaminophosphonium hexa-fluorophosphate), PyBOP (benzotriazole-1-yl-oxy-tris-pyrrolidinophosphonium hexafluoro-phosphate), PyBrOP (bromo-tris-pyrrolidinophosphonium hexafluorophosphate); via performed symmetrical anhydrides; via active esters such as pentafluorophenyl esters; or via performed HOBt (1-hydroxybenzotriazole) active esters or by using FMOC-amino acid fluoride and chlorides or by using FMOC-amino acid-N-carboxy anhydrides. Activation with HBTU (2-(1H-benzotriazole-1-yl),1,1,3,3-tetramethyluronium hexafluorophosphate) or HATU (2-(1H-7-aza-benzotriazole-1-yl),1,1,3,3-tetramethyluronium hexafluoro-phosphate) in the presence of HOBt or HOAt (7-azahydroxybenztriazole) is preferred.

The solid phase method can be carried out manually, although automated synthesis on a commercially available peptide synthesizer (e.g., Applied Biosystems 431A or the like; Applied Biosystems, Foster City, Calif.) is preferred. In a typical synthesis, the first (C-terminal) amino acid is loaded on the chlorotrityl resin. Successive deprotection (with 20% piperidine/NMP (N-methylpyrrolidone)) and coupling cycles according to ABI FastMoc protocols (ABI user bulletins 32 and 33, Applied Biosystems) are used to build the whole peptide sequence. Double and triple coupling, with capping by acetic anhydride, may also be used.

The synthetic peptides are cleaved from the resin and deprotected by treatment, for example, with TFA (trifluoroacetic acid) containing appropriate scavengers. Many such cleavage reagents, such as Reagent K (0.75 g crystalline phenol, 0.25 mL ethanedithiol, 0.5 mL thioanisole, 0.5 mL deionized water, 10 mL TFA) and others, can be used. The peptide is separated from the resin by filtration and isolated by ether precipitation. Further purification may be achieved by conventional methods, such as gel filtration and reverse phase HPLC (high performance liquid chromatography). Synthetic mimetics according to the present invention may be in the form of pharmaceutically acceptable salts, especially base-addition salts including salts of organic bases and inorganic bases. The base-addition salts of the acidic amino acid residues are prepared by treatment of the peptide with the appropriate base or inorganic base, according to procedures well known to those skilled in the art, or the desired salt may be obtained directly by lyophilization out of the appropriate base.

Generally, those skilled in the art will recognize that peptides as described herein may be modified by a variety of chemical techniques to produce compounds forming essentially the same immunological epitope as the unmodified peptide, and optionally having other desirable properties. For example, carboxylic acid groups of the peptide may be provided in the form of a salt of a pharmaceutically-acceptable cation. Amino groups within the peptide may be in the form of a pharmaceutically-acceptable acid addition salt, such as the HCl, HBr, acetic, benzoic, toluene sulfonic, maleic, tartaric and other organic salts, or may be converted to an amide. Thiols can be protected with any one of a number of well-recognized protecting groups, such as acetamide groups. Those skilled in the art will also recognize methods for introducing cyclic structures into the peptides of this invention so that the native binding configuration will be more nearly approximated. For example, a carboxyl terminal or amino terminal cysteine residue can be added to the peptide, so that when oxidized the peptide will contain a disulfide bond, thereby generating a cyclic peptide. Other peptide cyclizing methods include the formation of thioethers and carboxyl- and amino-terminal amides and esters.

Specifically, a variety of techniques are available for constructing peptide derivatives, analogues and mimetics with the same or similar desired immunological activity as the corresponding peptide compound but with more favorable activity than the peptide with respect to solubility, stability, and susceptibility to hydrolysis and proteolysis. Such derivatives, analogues and mimetics include peptides modified at the N-terminal amino group, the C-terminal carboxyl group, and/or changing one or more of the amido linkages in the peptide to a non-amido linkage. It will be understood that two or more such modifications can be coupled in one peptide mimetic structure (e.g., modification at the C-terminal carboxyl group and inclusion of a —CH₂— carbamate linkage between two amino acids in the peptide).

Amino terminus modifications include alkylating, acetylating, adding a carbobenzoyl group, and forming a succinimide group. Specifically, the N-terminal amino group can then be reacted to form an amide group of the formula RC(O)NH— where R is alkyl, preferably lower alkyl, and is added by reaction with an acid halide, RC(O)Cl or acid anhydride. Typically, the reaction can be conducted by contacting about equimolar or excess amounts (e.g., about 5 equivalents) of an acid halide to the peptide in an inert diluent (e.g., dichloromethane) preferably containing an excess (e.g., about 10 equivalents) of a tertiary amine, such as diisopropylethylamine, to scavenge the acid generated during reaction. Reaction conditions are otherwise conventional (e.g., room temperature for 30 minutes). Alkylation of the terminal amino to provide for a lower alkyl N-substitution followed by reaction with an acid halide as described above will provide for N-alkyl amide group of the formula RC(O)NR—. Alternatively, the amino terminus can be covalently linked to succinimide group by reaction with succinic anhydride. An approximately equimolar amount or an excess of succinic anhydride (e.g., about 5 equivalents) are used and the terminal amino group is converted to the succinimide by methods well known in the art including the use of an excess (e.g., ten equivalents) of a tertiary amine such as diisopropylethylamine in a suitable inert solvent (e.g., dichloromethane), as described in Wollenberg et al., U.S. Pat. No. 4,612,132, is incorporated herein by reference in its entirety. It will also be understood that the succinic group can be substituted with, for example, C₂- through C₆-alkyl or —SR substituents, which are prepared in a conventional manner to provide for substituted succinimide at the N-terminus of the peptide. Such alkyl substituents are prepared by reaction of a lower olefin (C₂- through C₆-alkyl) with maleic anhydride in the manner described by Wollenberg et al., supra., and —SR substituents are prepared by reaction of RSH with maleic anhydride where R is as defined above. In another advantageous embodiments, the amino terminus is derivatized to form a benzyloxycarbonyl-NH— or a substituted benzyloxycarbonyl-NH— group. This derivative is produced by reaction with approximately an equivalent amount or an excess of benzyloxycarbonyl chloride (CBZ-Cl) or a substituted CBZ-Cl in a suitable inert diluent (e.g., dichloromethane) preferably containing a tertiary amine to scavenge the acid generated during the reaction. In yet another derivative, the N-terminus comprises a sulfonamide group by reaction with an equivalent amount or an excess (e.g., 5 equivalents) of R—S(O)₂Cl in a suitable inert diluent (dichloromethane) to convert the terminal amine into a sulfonamide, where R is alkyl and preferably lower alkyl. Preferably, the inert diluent contains excess tertiary amine (e.g., ten equivalents) such as diisopropylethylamine, to scavenge the acid generated during reaction. Reaction conditions are otherwise conventional (e.g., room temperature for 30 minutes). Carbamate groups are produced at the amino terminus by reaction with an equivalent amount or an excess (e.g., 5 equivalents) of R—OC(O)Cl or R—OC(O)OC₆H₄-p-NO₂ in a suitable inert diluent (e.g., dichloromethane) to convert the terminal amine into a carbamate, where R is alkyl, preferably lower alkyl. Preferably, the inert diluent contains an excess (e.g., about 10 equivalents) of a tertiary amine, such as diisopropylethylamine, to scavenge any acid generated during reaction. Reaction conditions are otherwise conventional (e.g., room temperature for 30 minutes). Urea groups are formed at the amino terminus by reaction with an equivalent amount or an excess (e.g., 5 equivalents) of R—N═C═O in a suitable inert diluent (e.g., dichloromethane) to convert the terminal amine into a urea (i.e., RNHC(O)NH—) group where R is as defined above. preferably, the inert diluent contains an excess (e.g., about 10 equivalents) of a tertiary amine, such as diisopropylethylamine. Reaction conditions are otherwise conventional (e.g., room temperature for about 30 minutes).

In preparing peptide mimetics wherein the C-terminal carboxyl group is replaced by an ester (e.g., —C(O)OR where R is alkyl and preferably lower alkyl), resins used to prepare the peptide acids are employed, and the side chain protected peptide is cleaved with base and the appropriate alcohol, e.g., methanol. Side chain protecting groups are then removed in the usual fashion by treatment with hydrogen fluoride to obtain the desired ester. In preparing peptide mimetics wherein the C-terminal carboxyl group is replaced by the amide —C(O)NR₃R₄, where R₃ and R₄ are independently alkyl and preferably lower alkyl, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH₂). Alternatively, use of the chloromethylated resin during peptide synthesis coupled with reaction with ammonia to cleave the side chain Protected peptide from the support yields the free peptide amide and reaction with an alkylamine or a dialkylamine yields a side chain protected alkylamide or dialkylamide (i.e., the C-terminus is —C(O)NRR₁, where R and R₁ are alkyl and preferably lower alkyl). Side chain protection is then removed in the usual fashion by treatment with hydrogen fluoride to give the free amides, alkylamides, or dialkylamides.

In another alternative embodiment, the C-terminal carboxyl group or a C-terminal ester can be induced to cyclize by displacement of the —OH or the ester (—OR, where R is alkyl and preferably lower alkyl) of the carboxyl group or ester respectively with the N-terminal amino group to form a cyclic peptide. For example, after synthesis and cleavage to give the peptide acid, the free acid is converted in solution to an activated ester by an appropriate carboxyl group activator such as dicyclohexylcarbodiimide (DCC), for example, in methylene chloride (CH₂Cl₂), dimethyl formamide (DMF), or mixtures thereof. The cyclic peptide is then formed by displacement of the activated ester with the N-terminal amine. Cyclization, rather than polymerization, can be enhanced by use of very dilute solutions according to methods well known in the art.

Peptide mimetics as understood in the art and provided by the invention are structurally similar to the paradigm peptide of the invention, but have one or more peptide linkages optionally replaced by a linkage selected from the group consisting of: —CH₂NH—, —CH₂S—, —CH₂CH₂—, —CH═CH— (in both cis and trans conformers), —COCH₂—, —CH(OH)CH₂—, and —CH₂SO—, by methods known in the art and further described in the following references: Spatola, 1983, in CHEMISTRY AND BIOCHEMISTRY OF AMINO ACIDS, PEPTIDES, AND PROTEINS, (Weinstein, ed.), Marcel Dekker: New York, p. 267; Spatola, 1983, Peptide Backbone Modifications 1: 3; Morley, 1980, Trends Pharm. Sci. pp. 463-468; Hudson et al., 1979, Int. J. Pept. Prot. Res. 14: 177-185; Spatola et al., 1986, Life Sci. 38: 1243-1249; Hann, 1982, J. Chem. Soc. Perkin Trans. I 307-314; Almquist et al., 1980, J. Med. Chem. 23: 1392-1398; Jennings-White et al., 1982, Tetrahedron Lett. 23: 2533; Szelke et al., 1982, European Patent Application, Publication No. EP045665A; Holladay et al., 1983, Tetrahedron Lett. 24: 4401-4404; and Hruby, 1982, Life Sci. 31: 189-199, each of which is incorporated herein by reference. Such peptide mimetics may have significant advantages over polypeptide embodiments, including, for example: being more economical to produce, having greater chemical stability or enhanced pharmacological properties (such half-life, absorption, potency, efficacy, etc.), enhanced antigenicity, and other properties.

Mimetic analogs of the epitope peptides of the invention may also be obtained using the principles of conventional or rational drug design (see, Andrews et al., 1990, Proc. Alfred Benzon Symp. 28: 145-165; McPherson, 1990, Eur. J. Biochem. 189:1-24; Hol et al., 1989, in MOLECULAR RECOGNITION: CHEMICAL AND BIOCHEMICAL PROBLEMS, (Roberts, ed.); Royal Society of Chemistry; pp. 84-93; Hol, 1989, Arzneim-Forsch. 39:1016-1018; Hol, 1986, Agnew Chem. Int. Ed. Engl. 25: 767-778, the disclosures of which are herein incorporated by reference).

Kits as provided by the invention comprise antibodies of the invention, in embodiments that are polyclonal antisera, monoclonal antibodies or fragments or derivatives thereof, and instructions for their use. The components of the kit are advantageously provided in a container to preserve their integrity. In certain embodiments, the antibodies of the invention are provided in dry form, as powders or lyophilizates, and in these embodiments the kit advantageously includes liquid buffers or other reagents for reconstitution of the dry antibody preparations, as well as instructions for such reconstitution. Certain embodiments of the kits of the invention include reagents, in dried or liquid form, for use in the practice of the methods of the invention. These reagents can include, inter alia, buffers, salts, hybridization solutions, washing solutions, secondary antibodies, reagents for labeling primary or secondary antibodies, and reagents such as enzyme substrates for developing the results of, for example, an in situ hybridization assay. Instructions for use of any of these reagents are also advantageously included in such kits.

The description set forth above and the Examples set forth below recite exemplary embodiments of the invention. The following Examples are intended to further illustrate certain preferred embodiments of the invention and are not limiting in nature.

### EXAMPLES

### Example 1

### Preparation of Antigenic Peptide by Solid Phase Peptide Synthesis

An exemplary peptide (having the amino acid sequence: RQRREVHEDAHKQV; SEQ ID NO:1) provided by the invention for use as specific antigen for raising the anti-ρ1-GABA_{C} antibodies of the invention is prepared as follows.

Solid phase peptide synthesis (SPPS) is carried out on a 0.25 millimole (mmole) scale using an Applied Biosystems Model 431A Peptide Synthesizer and using 9-fluorenylmethyl-oxycarbonyl (Fmoc) amino-terminus protection, coupling with dicyclohexylcarbodiimide/hydroxybenzotriazole or 2-(1H-benzotriazol-1-yl)-1,1,3,3-tetramethyluronium hexafluoro-phosphate/hydroxybenzotriazole (HBTU/HOBT), and using p-hydroxymethyl phenoxymethyl-polystyrene (HMP) resin or Sasrin™, or chlorotrityl resin for carboxyl-terminus acids or Rink amide resin for carboxyl-terminus amides.

Sasrin™ resin-bound peptides are cleaved using a solution of 1% TFA in dichloromethane to yield the protected peptide. Where appropriate, protected peptide precursors are cyclized between the amino- and carboxyl-termini by reaction of sidechain-protected, amino-terminal free amine and carboxyl-terminal free acid using diphenylphosphorylazide.

HMP or Rink amide resin-bound products are routinely cleaved and protected cyclized peptides deprotected using a solution comprised of trifluoroacetic acid (TFA), or TFA and methylene chloride, optionally comprising water, thioanisole, ethanedithiol, and triethylsilane or triisopropylsilane in ratios of 100:5:5:2.5:2, for 0.5-3 hours at room temperature. Where appropriate, products were re-S-tritylated in triphenolmethanol/TFA, and N-Boc groups re-introduced into the peptide using (Boc)₂O.

Crude peptides are purified by preparative high pressure liquid chromatography (HPLC) using a Waters Delta Pak C18 column and gradient elution using 0.1% trifluoroacetic acid (TFA) in water modified with acetonitrile. Acetonitrile is evaporated from the eluted fractions which are then lyophilized. The identity of each product is confirmed by fast atom bombardment mass spectroscopy (FABMS) or by electrospray mass spectroscopy (ESMS).

### Example 2

### Preparation of Polyclonal Antibodies

Polyclonal antibodies specific for the ρ1-GABA_{C} receptor protein species are prepared using the epitopic peptide disclosed in Example 1. Polyclonal antibodies against an oligopeptide of SEQ ID NO:1 prepared according to Example 1, or against purified recombinant peptide of SEQ ID NO:1, were generated in guinea pigs according to standard procedures well known in the art (see, for example, Harlow & Lane, Id.). Specifically, purified peptides were conjugated with keyhole limpet hemocyanin (KLH) using conventional methods (Harlow & Lane, Id.) after the addition of a carboxyl-terminal cysteine residue to the peptide of SEQ ID NO: 1.

Antibodies produced using this method were purified as follows. First, IgG was purified from guinea pig serum employing the technique of affinity chromatography using protein A-Sepharose. In this protocol, protein A-Sepharose CL-4B beads (0.3 g/column, obtained from Sigma Chemical Co., St. Louis, Mo.) were prepared by swelling in a solution of 0.1 M Tris-base, pH 8.0 for 30 min at 22° C. The beads were then added to a column and washed with 60 mL of 0.1 M Tris-base, pH 8.0. To the washed beads, a mixture of 1.7 mL guinea pig serum with 189 μL of 1 M Tris-base, pH 8.0 was added. Columns were incubated on an automated rocker for 16 hr at 4° C. Following incubation, the column was again washed, first with 10 mL of 0.1 M Tris-base, pH 8.0 and then with 10 mL of 0.01 M Tris-base, pH 8.0. Next, IgG was eluted using 8 mL glycine (100 mM, pH 3.0) and 500 μL fractions were collected with the addition of 50 μL of 1 M Tris-base, pH 8.0 to each 500 μL fraction to neutralize the pH. Absorbance of eluted fractions was read at 280 nm and the samples with the highest absorbance, representing eluted IgG, were pooled for further processing.

Peptide-specific antibody (anti-GABA_{C} IgG) was purified from total guinea pig serum IgG (obtained through protein A-Sepharose chromatography as described above) using affinity column chromatography in which column-bound N-14 peptide (SEQ ID NO: 1) served as ligand (Khasawneh et al., 2006 J. Biol. Chem. 281:26951-26965). This chromatography used Affi-Gel 10 beads (Bio-Rad Laboratories, Hercules, Calif.), containing a N-hydroxysuccinimide ester of a derivatized crosslinked agarose support with high capacity for selectively purifying proteins with a free alkyl or aryl amino group. The Affi-Gel 10 (1 mL prior to suspension) beads were first washed with 10 mL cold isopropanol. Washed beads were then mixed with 2 mL of a 2.5 mg/mL solution of N-14 peptide (SEQ ID NO: 1) (in 100% DMSO) for 4 hr at 4° C. Columns were next drained and washed with 6 mL phosphate buffered saline (0.039 M NaH₂PO₄, 0.061 M Na₂HPO₄, 0.14 NaCl, 0.02% NaN₃, pH 7.4; PBS). An IgG solution comprising pooled fractions of protein Sepharose-A purified IgG was then added and the beads incubated on an automated rocked for 16 hr at 4° C. The column was next washed with 9 mL PBS and specifically bound antibodies immediately eluted by the addition of 4 mL glycine (100 mM, pH 2.5). Next, 500 μL fractions were collected with the addition of 50 μL of 1 M Tris-base, pH 8.0 to each 500 μL fraction to neutralize the pH. The samples with the highest absorbance (at 280 nm) readings were pooled, and then dialyzed in 4 L PBS for 16 hr at 4° C. Columns were washed with 6 mL PBS and stored in the same solution containing 0.02% NaN₃. The final concentration of the affinity-purified antibody (henceforth referred to as GABA_{C} Ab N-14) was 0.24 mg/mL.

### Example 3

### Characterization ρ1-GABA_{C} Polyclonal Antisera

The polyclonal antisera comprising antibodies specific for human ρ1-GABA_{C} were characterized as follows.

Immunoblotting experiments were performed using affinity-purified anti-human ρ1-GABA_{C} (prepared as set forth in Examples 1 and 2 above, termed “GABA_{C} Ab N-14” herein) as a primary antibody in Western blot procedures at a dilutions of between 1/7,000 to 1/10,000. Secondary antibody (HRP-conjugated, goat-anti-guinea pig antibody, obtained from Santa Cruz Biotechnology Inc., Santa Cruz, Calif.) was used at a 1/5,000 dilution. Spot blotting was performed using N-14 peptide (SEQ ID NO: 1) dotted on a PVDF membrane, and probed with either (i) affinity-purified GABA_{C} Ab N-14, followed by the secondary antibody, or (ii) secondary antibody only (i.e., omitting the GABA_{C} Ab N-14). The results of these experiments are shown in FIG. 2A. Peptide spots yield a strong signal when assayed with the affinity purified GABA_{C} Ab N-14 (FIG. 2A, lane 1) that was not detected in the absence of GABA_{C} Ab N-14 (FIG. 2A, lane 2). These data illustrate recognition/reactivity of the affinity-purified antibody with its cognate peptide.

Western blot experiments were performed on whole cell lysates prepared from the following cellular sources: (i) Neuroblastoma cell lines, stably transfected to express the human ρ1 GABA_{C} receptor (SHp5-human ρ1, gift from Dr. David S. Weiss, University of Texas Health Science Center at San Antonio, San Antonio, Tex.); and (ii) non-expressing neuroblastoma cells as controls (SHSY5Y, obtained from the American Type Culture Collection (ATCC), Manassas, Va.). Western blot experiments were also performed using lysates made from Xenopus laevis oocytes, using membrane protein from either the control non-expressing oocytes or oocytes transfected to express the human ρ1 GABA_{C} receptor (Qian et al., 1997 Vis. Neurosci. 14: 843-851; Vu et al., 2005 Biomaterials 26: 1895-1903; Gussin et al., 2006 J. Am. Chem. Soc. 128:15701-15713), using the method described by Wible et al. 1998 J. Biol. Chem. 273:11745-11751. For all whole-cell lysate and membrane protein preparations of the investigated neuroblastoma cells and oocytes, the amount of protein was normalized at 15-25 μg per lane.

Western blot assays were performed using four separate conditions: (1) cells expressing human GABA_{C} probed with the affinity-purified GABA_{C} Ab N-14, followed by the secondary antibody, (2) non-expressing cells probed with the affinity-purified GABA_{C} Ab N-14, followed by the secondary antibody, (3) cells expressing human GABA_{C} probed only with the secondary antibody (affinity-purified GABA_{C} Ab N-14 omitted) (first control), and (4) cells expressing human GABA_{C} probed with the affinity-purified GABA_{C} Ab N-14 pre-absorbed with N-14 peptide (SEQ ID NO: 1) (3 μg/ml, 30 min, RT), followed by the secondary antibody (second control).

Results obtained from neuroblastoma cell preparations are shown in FIG. 2B and results for oocyte membrane protein preparations in FIG. 2C under condition (1) (see above). These results showed the presence of a single band at approximately 55 kDa, the expected molecular weight of a single human GABA_{C} ρ1 subunit. This ˜55 kDa band was not present in cells that did not express the GABA_{C} receptor (condition (2) described above). Omission of the affinity-purified GABA_{C} Ab N-14 as a primary antibody (condition 3) led to the loss of the ˜55 kDa band. Pre-absorption of the affinity-purified GABA_{C} Ab N-14 with N-14 peptide (SEQ ID NO: 1) (condition 4) also resulted in the loss of the ˜55 kDa band.

Western blot analyses were also performed using rat retina and rat brain cell lysates (20 μg/lane), as shown in FIG. 2D. Bands at molecular weight ˜55 kDa were observed in lanes corresponding to rat retina, and to a lesser extent to rat brain, when probed with GABA_{C} Ab N-14 (FIG. 2D). Consistent with the pattern observed in cell line preparations, the exclusion of GABA_{C} Ab N-14 and the pre-absorption of GABA_{C} Ab N-14 with N-14 resulted in the loss of the ˜55 kDa band in each case. These data also demonstrated that GABA_{C} Ab N-14 was immunologically cross-reactive with rat GABA_{C} receptors, as expected in view of their sequence similarity (see above).

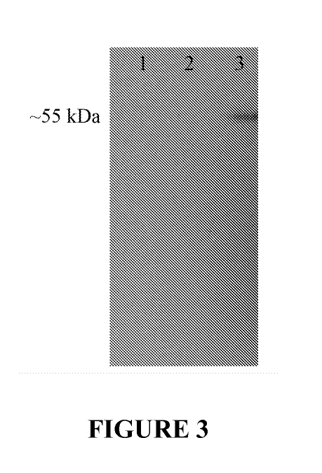

In order to investigate antibody specificity for GABA_{C} receptor subtypes, the reactivity of GABA_{C} Ab N-14 with GABA_{A} receptors was also tested by Western blotting. GABA_{A} receptors, like GABA_{C}, are ligand-gated ion channels; however their subunit composition and their pharmacological properties are distinct from those of GABA_{C}. Membrane proteins from oocytes expressing the human α1β2γ2 GABA_{A} receptor were prepared and probed with GABA_{C} Ab N-14, under condition (1) described above for GABA_{C} expressing oocytes. FIG. 3 shows that the ˜55 kDa band, present for GABA_{C} (lane 3), is absent from the GABA_{A} lane (lane 1) as well as from the non-expressing control lane (lane 2). These data demonstrated the specificity of GABA_{C} Ab N-14, which is immunoreactive with human GABA_{C} receptors but not with GABA_{A} receptors.

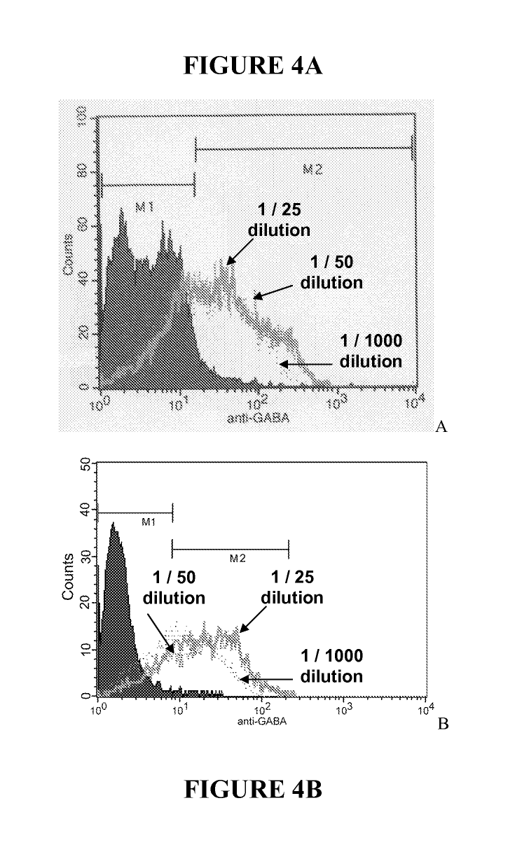

The GABA_{C} Ab N-14 antisera was further characterized by flow cytometry performed on GABA_{C}-expressing neuroblastoma cells (SH5p-human ρ1 cells) and on non-expressing control neuroblastoma cells (SHSY5Y), using a 1/25 to 1/1,000 dilution of affinity-purified GABA_{C} Ab N-14 as a primary antibody, and a FITC-labeled goat-anti guinea pig IgG (Santa Cruz Biotechnology), 1/50 dilution as secondary antibody. Two separate control experiments were performed: experiments in which non-immunized guinea-pig IgG was substituted for the primary antibody; and experiments which omitted primary antibody but included secondary antibody. These results are shown in FIG. 4. The results revealed a rightward shift in the mean fluorescence intensity of GABA_{C}-expressing neuroblastoma cells probed with affinity-purified GABA_{C} Ab N-14. Specifically, when the GABA_{C}-expressing cells were probed with affinity-purified GABA_{C} Ab N-14 at 1/25 to 1/1,000 dilution, the shift corresponded with the presence of approximately 63 to 47% positive cells (FIG. 4). By comparison, no significant shift was observed when SHp5-ρ1 cells were probed with secondary antibody only (absence of guinea pig IgG), or when the non-GABA_{C} expressing SHSY5Y cells were probed with affinity-purified GABA_{C} Ab N-14.

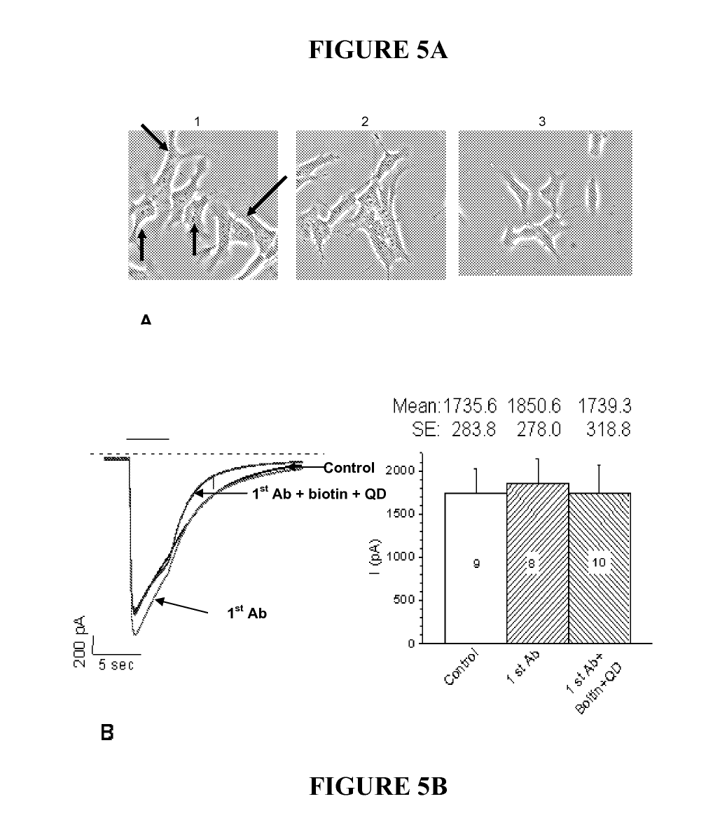

GABA_{C} Ab N-14 antisera was also characterized by immunofluorescence labeling of live neuroblastoma cells, using a 1/1000 to 1/2,000 dilution of affinity purified GABA_{C} Ab N-14 as a primary antibody. These results are shown in FIG. 5A, where Panel 1 shows results obtained when the secondary antibody used for detection was biotin-labeled, goat-anti-guinea pig IgG (1/400), followed by incubation with streptavidin-quantum dots (SA-qdot) at a 10 nM concentration (Invitrogen, Carlsbad, Calif.). Similar results were obtained using FITC-labeled goat-anti guinea pig IgG (Santa Cruz) at 1/400 dilution as secondary antibody (data not shown). As shown by Panel 2 of FIG. 5A, no labeling of GABA_{C}-expressing neuroblastoma cells was observed when the GABA_{C} Ab N-14 primary antibody was omitted. Treatment with GABA_{C} Ab N-14 as primary antibody, and subsequent treatment with biotinylated secondary antibody and streptavidin-coated quantum dots, did not yield a fluorescence signal in non-expressing neuroblastoma cells (Panel 3 of FIG. 5A).

Finally, electrophysiological testing of the GABA-induced response in the GABA_{C}-expressing neuroblastoma cells, following labeling with GABA_{C} Ab N-14, the biotinylated secondary antibody, and SA-qdot, was performed (results shown in FIG. 5B). Under these conditions, the response of the cells to 10 μM GABA was not significantly altered by the antibody labeling procedure.

It should be understood that the foregoing disclosure emphasizes certain specific embodiments of the invention and that all modifications or alternatives equivalent thereto are within the spirit and scope of the invention as set forth in the appended claims.

We claim:

1. An antibody that specifically binds to ρ1-GABA_{C} protein.

2. An antibody of claim 1 comprised in a polyclonal antisera.

3. An antibody of claim 1 that is a monoclonal antibody.

4. An antibody of claim 1 that specifically binds to an epitope from the amino acid sequence identified by SEQ ID NO: 1.

5. An antibody of claim 1 raised by immunizing an animal with a peptide having the amino acid sequence is identified by SEQ ID NO: 1.

6. An antibody of claim 1 wherein the ρ1-GABA_{C} protein is expressed in retinal cells.

7. A method for detecting ρ1-GABA_{C} protein comprising the steps of contacting a sample comprising ρ1-GABA_{C} protein with an antibody of claim 1 and detecting binding of the antibody with the protein.

8. A method of claim 7, wherein the ρ1-GABA_{C} protein is expressed in retinal cells.

9. A method of claim 7, wherein the ρ1-GABA_{C} protein is detected in a tissue sample.

10. A method of claim 9, wherein the ρ1-GABA_{C} protein is detected by in situ immunohistochemistry.

11. A method of claim 7, wherein the ρ1-GABA_{C} protein is detected by Western blot analysis.

12. A method for detecting expression of ρ1-GABA_{C} protein comprising the steps of contacting ρ1-GABA_{C} protein with an antibody of claim 1 and detecting binding of the antibody with the protein.

13. A method of claim 12, wherein the ρ1-GABA_{C} protein is expressed in retinal cells.

14. A method of claim 12, wherein the ρ1-GABA_{C} protein is detected in a tissue sample.

15. A method of claim 14, wherein the ρ1-GABA_{C} protein is detected by in situ immunohistochemistry.

16. A method of claim 12, wherein the ρ1-GABA_{C} protein is detected by Western blot analysis.

17. A method according to any of claims 1 through 16, wherein the ρ1-GABA_{C} protein is human ρ1-GABA_{C} protein.

18. A kit comprising a preparation of an antibody according to claim 1 and instructions.

19. A kit according to claim 18, further comprising reagents for performing an immunological assay.

20. A kit according to claim 18 or 19, wherein the antibody is immunologically specific for human ρ1-GABA_{C} protein.
